(19) United States
(12) Patent Application Publication
(10) Pub. No.: US 2022/0275719 A1
(43) Pub. Date: Sep. 1, 2022

(54) METHOD AND SYSTEM FOR A FAST AND ACCURATE ESTIMATION OF PETROPHYSICAL PROPERTIES OF ROCK SAMPLES

(71) Applicant: Khalifa University of Science and Technology, Abu Dhabi (AE)

(72) Inventors: Ali ALSUMAITI, Abu Dhabi (AE); Abdul Ravoof SHAIK, Chester Hill, NSW (AU); Abdul Khader JILANI, Vijayawada, Andhra Pradesh (IN); Moussa TEMBELY, Abu Dhabi (AE); Waleed Salem ALAMERI, Abu Dhabi (AE)

(21) Appl. No.: 17/632,051

(22) PCT Filed: Jul. 20, 2020
(86) PCT No.: PCT/IB2020/056792

§ 371 Date: Feb. 1, 2022

Related U.S. Application Data
(60) Provisional application No. 62/881,595, filed on Aug. 1, 2019.

(51) Int. Cl. E21B 47/002; G01N 33/24; G06T 7/00

(52) U.S. Cl. CPC E21B 47/002; G01N 33/241; G06T 7/0002; G06T 2207/10081; G06T 2207/20084; G06T 2207/20081

(57) ABSTRACT

There is provided a system and process to predict the petrophysical properties of unclean rock samples using Medical-CT scanned three-dimensional (3D) images at both low and high resolutions. The captured 3D images are passed through machine learning, statistical methods and data lookups to identify the petrophysical properties of rock samples. Also disclosed is the process of measuring phase saturations of a clean rock sample or porous medium using Micro-CT scanned three-dimensional (3D) images.

### FIELD OF THE INVENTION

The present invention relates to the field of Petro Physics, more particularly to the prediction of petrophysical properties of rock samples.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

Petrophysics refers to a branch of geology which deals with the physical properties and behavior of rocks. A major application of Petrophysics is to study reservoirs for the hydrocarbon industry. Some of the major petrophysical properties studied are lithology, porosity, permeability, water saturation and formation density.

A core plug is a sample of rock in the shape of a cylinder. Plugs could be taken from the side of a drilled oil or gas well. Alternatively, multiple core plugs, or small cylindrical samples can be extracted from a whole core well. These core plugs are then dried and measured to define the porosity and permeability of the reservoir rock, fluid saturation and grain density. Formation porosity and permeability are usually measured in the laboratory from core plugs or estimated from well logs and well test data. However, core analysis could be enhanced utilizing whole core material or old samples. Recently, machine learning has been used to tackle industrial applications ranging from engineering problems to medical diagnostics. Nevertheless, in petroleum engineering, most of the machine learning applications are concerned with reservoir characterization, estimating petrophysical properties from wells log, rock typing, and, very recently, drilling optimization. Currently, existing technologies used in direct estimation of porosity and permeability require the samples to be uniformly cylindrical. Utilizing uncleaned complex carbonate core plugs from medical-CT images as well as micro CT images will definitely impact the accuracy of such measurements. In order to perform routine or special core analysis (RCA/SCAL) measurements, reservoir core plugs must undergo the time-consuming cleaning process, which might be ineffective, in some cases, to restore original core properties. The proposed method aims to estimate petrophysical properties such as porosity and permeability based on the computed tomography image of dry unclean samples.

Micro and Medical CT imaging techniques are used to acquire 3D images that reveal the rock structure and the suitability for lab measurements. Micro-CT offers advantages in terms of image resolution (down to 0.5 μm) to capture the pore space, however it has a number of limitations like artifacts arising from the polychromatic nature of the X-ray preventing the distinction of materials and the Micro-CT is limited for scanning smaller samples. Higher resolution limits the Micro-CT usage to smaller samples, less representative of the core plug. The medical-CT imaging rectifies the limitation of Micro-CT by enabling the scan for larger samples compared to micro-CT, however medical-CT provides qualitative characterization of the plug, whereas the estimation of Petrophysical properties such as porosity and permeability relies on the lab measurement. Thus at present, there is inadequate techniques to estimate both porosity and permeability from Medical-CT images of unclean rock samples.

Dual-Energy-CT scan (DE-CT) scan was developed initially for application in the petroleum field at core scale. The rock samples were scanned at two energy levels (high and low) including proper calibration materials. The high energy images are more sensitive to bulk density while the low energy images are more sensitive to the mineralogy. By evaluating the attenuation coefficients, at the 2-energy levels, it is possible to estimate through empirical equations the effective atomic number, electronic density and porosity of the scanned samples. However, the choice of appropriate energy levels and the parameters for attenuation equations are not obvious. In addition, DE-CT cannot predict the sample permeability because the permeability is inferred based on empirical correlation with the porosity, however this approach is invalid for complex carbonates, where there is no explicit correlation between porosity and permeability. In addition, porosity can be also estimated by comparing the image of a dry and clean sample before and after saturation. However, such technique requires cleaning the core sample

Recently, digital rock physics (DRP) which relies on micro-CT images was used as a tool of choice to estimate rock porosity and permeability at pore-scale, however its accuracy and focus on small size sample (0.5 mm) make its predictive capability insufficient to infer on the properties at core-scale (38 mm). In addition, the upscaling challenge inherent to DRP—from pore-scale (um) to core-scale (cm)—is still to be fully addressed. Currently, machine learning techniques are mostly applied to micro-CT images to estimate petrophysical properties at pore-scale. The present method aims to estimate unclean reservoir rock porosity and permeability from medical-CT images without any computation of physical processes, as the pore structure (0.5 μm) is inaccessible at the CT image resolution (100 μm).

Estimating trapped phase saturations is helpful in predicting total recoverable oil from the reservoir. This information is essential for hydrocarbon field development plans. Various techniques have evolved in the past to estimate the trapped phase content. Typically, in oil and gas industry, the oil content is expressed as a percentage of the volume of the pore space existing in the porous medium, which is called oil saturation and the other saturation phases could include either water and gas or water only where the sum of saturation is equal to unity. There are various well established tertiary recovery techniques in oil and gas industry, such as chemical enhanced oil recovery or gas EOR, are used to recover further oil from the reservoir. The trapped oil cannot be recovered when capillary force exceeds the gravity or viscous forces. The degree to recover trapped oil depends on size of ganglion in relative to the viscous and (or) gravity forces. Some amount of oil can be trapped into the porous medium which cannot be recovered is called ‘irreducible oil saturation’. It is worth noting that, failure to accurately estimate residual oil saturation can affect the recoverable hydrocarbon reserves and ultimately reservoir productivity. Previously, various well-established techniques were presented in literature to estimate the residual oil saturation of a rock sample. Recent advances in reservoir characterization has led to study in-situ saturation with techniques ranging from NMR, gamma ray to incorporation of medical and (or) micro-CT to core flooding setup. In addition, various efforts have been done in the past to successfully visualize and characterize oil movement inside the rock sample dynamically. However, it is worth noting that all the aforementioned procedures are time consuming.

Currently, there is a need for fast and accurate estimation method to determine petrophysical properties.

### SUMMARY OF THE INVENTION

Therefore, the objective of the present invention is to present a new methodology to accurately estimate petrophysical properties of unclean rock samples which will help identify and predict hydrocarbon reservoir volume.

The present invention involves a method of detecting a plurality of petrophysical properties of an uncleaned rock sample, the method comprising the steps of capturing an image of the uncleaned rock sample, passing the captured image through a feature extraction engine, providing an output from the feature extraction engine to a neural network for estimating the petrophysical properties of the uncleaned rock sample and displaying the estimated petrophysical properties of the uncleaned rock sample on a display medium.

In another embodiment, the image of the uncleaned rock sample is a three-dimensional (3D) image.

In another embodiment, the image of the uncleaned rock sample is a computed tomography (CT) image.

In another embodiment, the image of the uncleaned rock sample is a medical-CT image.

In another embodiment, the image of the cleaned rock sample is a micro CT image.

In another embodiment, the image of the uncleaned rock sample is acquired at either a low resolution or a high resolution.

In another embodiment, the micro-CT image is captured from a core-flooding equipment through a fluid displacement test.

In another embodiment, the petrophysical and SCAL properties comprise porosity, permeability, elastic property, relative permeability or capillary pressure.

In another embodiment, the feature extraction engine is a porosity-permeability predictor engine.

In another embodiment, the feature extraction engine is trained using an Out of the Box (OOTB) feature extractor and a pore network correction engine (PNCE).

In another embodiment, the Out of the Box (OOTB) feature extractor extracts features comprising porosity, pore volume distributions and pore size distributions of rock sample.

In another embodiment, the pore network Correction Engine (PNCE) computes permeability from binary images using a machine learning algorithm.

As another aspect of the present invention, a process for predicting saturation of oil within a reservoir is disclosed, the process comprising capturing an image of a porous medium obtained from the reservoir, passing the captured image through a feature extraction engine; and providing an output from the feature extraction engine to a neural network for estimating the petrophysical properties of the porous medium, wherein estimating the petrophysical properties of porous medium leads to prediction of the saturation of oil within the reservoir.

In another embodiment, the uncleaned porous medium is an uncleaned rock sample.

In another embodiment, the image of the uncleaned porous medium is a three-dimensional (3D) image.

In another embodiment, the image of the uncleaned porous medium is a computed tomography (CT) image.

In another embodiment, the image of the cleaned porous medium is a micro-CT image.

In another embodiment, the image of the uncleaned porous medium is acquired at either a low resolution or a high resolution.

In another embodiment, the micro-CT image is captured from a core-flooding equipment through a fluid displacement test.

In another embodiment, the petrophysical and SCAL properties comprise porosity, permeability, elastic property, relative permeability or capillary pressure.

In another embodiment, the feature extraction engine is a porosity-permeability predictor engine.

In another embodiment, the feature extraction engine is trained using an Out of the Box (OOTB) feature extractor and a pore network correction engine (PNCE).

In another embodiment, the Out of the Box (OOTB) feature extractor extracts features comprising porosity, pore volume distributions and pore size distributions of rock sample.

In another embodiment, the pore network Correction Engine (PNCE) computes permeability of the uncleaned porous medium using a machine learning algorithm.

### BRIEF DESCRIPTION OF THE DRAWINGS

The subject matter that is regarded as the invention is particularly pointed out and distinctly claimed in the claims at the conclusion of the specification. The foregoing and other aspects, features, and advantages of the invention are apparent from the following detailed description taken in conjunction with the accompanying drawings in which—

FIG. 1 depicts the logical diagram for the estimation of phase saturations using image processing performed on the scanned Micro-CT images.

FIG. 2 depicts the logical diagram for the prediction of phase saturations using features extracted from computer algorithms in accordance with the present invention.

FIG. 3 depicts a workflow for the prediction of petrophysical properties using neural network models in accordance with the present invention.

### DETAILED DESCRIPTION OF THE INVENTION

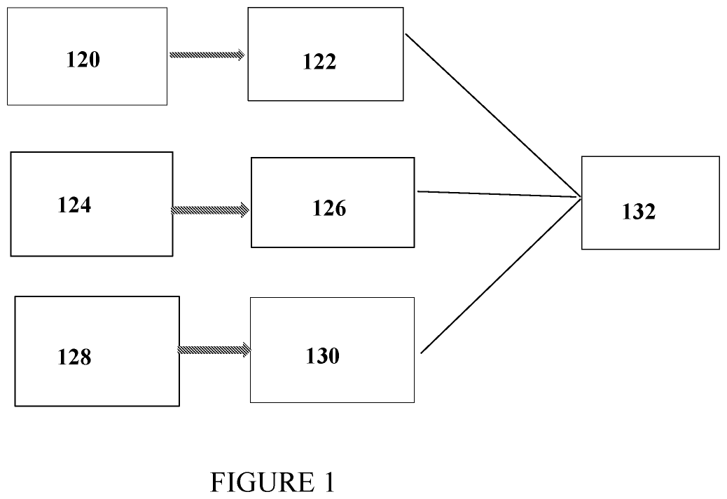

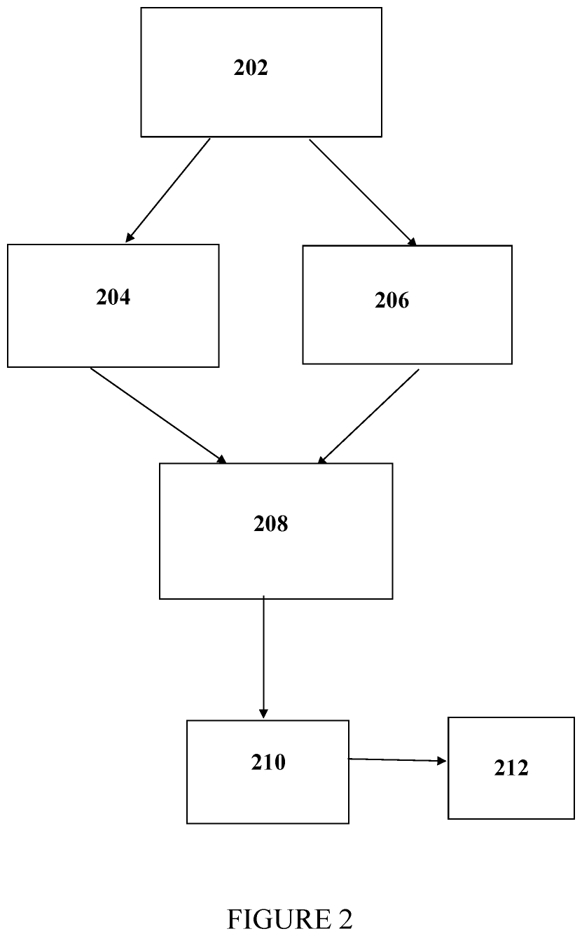

The aspects of the method or system to provide a system and method for fast and accurate estimation of petrophysical properties of unclean rock samples and identifying and predicting oil reservoir volume according to the present invention, will be described in conjunction with FIGS. 1-2. In the Detailed Description, reference is made to the accompanying figures, which form a part hereof, and in which is shown by way of illustration specific embodiments in which the invention may be practiced. It is to be understood that other embodiments may be utilized and logical changes may be made without departing from the scope of the present invention. The following detailed description, therefore, is not to be taken in a limiting sense, and the scope of the present invention is defined by the appended claims.

Predicting petrophysical and SCAL properties are essential in reservoir descriptions with direct impact on improved oil recovery (IOR), enhance oil recovery (EOR) strategy, completion designs, and reservoir management. The present invention relates to a system to detect in situ trapped phase saturations of porous medium and more particularly, to identifying initial oil saturation, remaining oil saturation and residual oil saturation. This involves using machine learning and statistical methods to measure properties of rock samples to quantify the parameters that indicate a rich hydrocarbon reserve. All these properties are predicted using a system that collects rock samples and takes micro CT images at a set resolution and orientation of the rock samples. Once this is done, the images and metadata of the samples are passed through machine learning (ML) models and data lookups to identify and predict the aforementioned properties. Phase saturations of oil, gas or water may be predicted.

In another embodiment, the present invention discloses a system and process to predict fast and accurately petrophysical properties from CT images acquired at both low and high resolutions. In particular, a process relying on machine and deep learning to measure the porosity and permeability of dry uncleaned rock samples scanned with medical-CT is disclosed. The system takes features such as formation top and bottom depth, average CT number, and 3D images as inputs—to predict both the porosity and permeability for a given formation.

In accordance with an embodiment depicted in FIG. 1, Micro-CT based core experiments are designed to estimate the trapped phase saturations. Initially, 100% brine saturated core plug 120 are placed inside the core holder and drainage core flooding experiment 124 is performed and core plug 120 is initialized either at ambient or reservoir conditions. The plug 120 is scanned and microCT image 122 is generated at initial stage. Image processing is performed on the scanned micro CT images and phase (oil/gas/water) saturations at various states are estimated.

The proposed invention describes the system and method for identifying and predicting hydrocarbon reservoir volume by detecting the in-situ trapped phase saturations of rock samples using features extracted from computer vision algorithms and further validated using previous historical data. In the present invention, the porosity and permeability of rock samples are estimated by scanning the rock samples using Medical-CT at low resolution without the need of cleaning the core. Further a hybrid network built on Convolutional neural network (CNN) and Deep Neural Network (DNN) is trained and validated with Medical-CT images of different test samples to estimate the porosity and permeability of the core sample. Plugs could be taken from the side of a drilled oil or gas well. Alternatively, multiple core plugs, or small cylindrical samples can be extracted from a whole core well. These core plugs are cleaned then dried and measured to define the porosity and permeability of the reservoir rock, fluid saturation and grain density. In order to perform special core analysis measurements, the reservoir core plugs must undergo the time consuming cleaning process, which might be ineffective in some cases.

An estimation of Petrophysical properties of core samples helps to identify the hydrocarbon reservoir volume, initial oil saturation, remaining oil saturation, residual oil saturation. Predicting petro physical properties is essential for reservoir management, completion designs, improved oil recovery (IOR) and enhance oil recovery (EOR) Strategy. The present invention involves using machine learning and statistical methods to measure properties of rock samples that helps to estimate hydrocarbon reserve.

In the present invention, the petrophysical properties are predicted using a system that collects rock samples and takes Micro-CT images at a set resolution and orientation of the rock samples. Further, the acquired images and metadata of the samples are passed through machine learning (ML) models and data lookups to identify and predict the aforementioned properties.

In accordance with the present invention, FIG. 2 depicts a logical diagram for the measurement of phase saturations output 212 using features extracted from computer algorithms and further validated from historical data to predict the phase saturations of the rock samples. 100% brine saturated Micro-CT Images 202 are captured from a standard Micro-CT based core-flooding equipment, through a fluids displacement test. The micro-CT core flooding experiment involves placing 100% brine saturated core samples inside a core holder and performing a drainage core flooding experiment to initialize the core plug at ambient or reservoir conditions. Further, the core plug is scanned and 100% brine saturated Micro-CT images 202 are generated.

In accordance with the present embodiment, the scanned 100% brine saturated Micro CT images 202 are passed through feature extraction engines, an Out of the Box (OOTB) feature extractor 204, consisting of a Pore Network Correction Engine (PNCE) (not shown) and a Deep Feature Extractor 206. The OOTB feature extractor 204 is built using algorithms, which extracts features such as porosity, pore volume distributions, pore size distributions and pore networks. All the features extracted from Micro-CT images 202 are three-dimensional and provides or displays the material constituency of the rock samples in a display medium. The Pore Network correction engine (PNCE) corrects the fast prediction of permeability obtained by pore network model (PNM) to obtain a more accurate estimation of permeability. The predictive ability of the pore network approach is used for computing the properties of the porous media. However, this is insufficient as the pore network approach relies on simple geometries.

Considering the Out of the Box Feature extractor 204, this engine is built using algorithms which will extract features such as porosity, pore volume distributions, pore size distributions, pore networks. In addition, for medical-CT images, features including CT number, formation top and bottom depth, raw and binary images are extracted. All the features are extracted from (micro or medical) CT images which are 3-dimensional and provide the material constituency. These features are typically the ones which have more predictive power in terms of the dependent variables.

With respect to the Pore Network Correction Engine (PNCE), while the pore network approach is the tool of choice and widely used for computing the properties of porous media, since it relies on simplified geometries, its predictive ability is insufficient. Unlike pore network model (PNM), voxel-based direct simulation is very accurate but quite resource intensive. To take advantage of the efficient computation provided by PNM and the accuracy of direct simulation, a machine-learning algorithm is developed to infer on the permeability of rock image scanned at high resolution. The relevant features, such as the porosity, the formation factor, and the permeability according to PNM, in addition to the 3D images, are fed into both a supervised machine learning model and a deep neural network to compute the permeability at the accuracy of voxel-based simulation such as lattice Boltzmann simulation. This engine corrects the fast prediction by pore network model (PNM) to a more accurate estimation of the permeability. This engine is based on thousands of segmented micro-CT images 202 at high resolution. The engine relies on machine and deep learning algorithms such as linear regression, gradient boosting, and physics-informed convolutional neural networks (CNNs).

Further, considering the Deep Feature extractor 206 in accordance with the present invention, this is a feature extractor which is built on deep neural networks. Convolutions neural networks are used to ingest the 3D image matrices and provide a vector representation of the 3D image that acts as a feature set for the predictions.

The 3D images and relevant features such as porosity and permeability according to PNM (Pore Network Model) are fed into PNCE model relying on supervised machine learning and a deep neural network, to compute accurate permeability. The PNCE engine is based on segmented Micro-CT images 202 at high resolution and depends on machine and deep learning algorithms such as linear regression, gradient boosting and convolutional neural networks.

In another embodiment, the 100% brine saturated Micro-CT images 202 are fed through the deep feature extractor 206, which relies on convolution neural networks and deep neural networks. Convolution neural network provides a vector representation of the 3D image that acts as a feature set for the predictions. Further the output of the feature extraction engines are passed through a material classifier block 208 which performs material classification. This group of artificial intelligence (AI) based classification models 208 have been trained on features from deep feature extractors 206 and OOTB 204. The material classifier 208 identifies the material of the rock sample and helps to weed out the anomalies in the system.

The material classifier 208 is built by running multiple models like random forest, neural networks and Meta learner, which is trained on top of the outputs for added accuracy. The material classifier identifies the goodness of the sample, if this classifier value is not same as the lookup value, then there is an issue with the sample or there is an anomaly. In accordance with the present embodiment, an output from the material classifier 208 is passed through an AI-based processor 210 in order to estimate phase saturations of the rock sample under test.

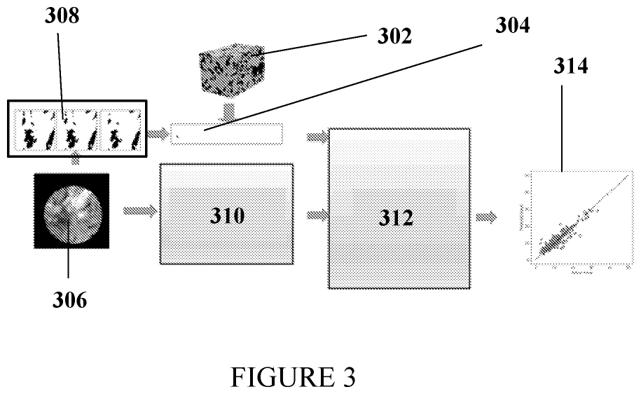

In accordance with the present invention, FIG. 3 depicts a workflow for the estimation of petrophysical properties using neural networks. A physically-based deep learning model is built on Convolutional Neural Network (CNN) 310 and Deep Neural Network (DNN) 312. The convolutional neural network (CNN) 310 takes raw medical-CT images 308 as input and deep neural network (DNN) 312 takes features such as sample depth, CT number, factious porosity and permeability as input. This hybrid network is trained and validated with Medical-CT images 308 of different samples to output a final porosity and permeability value of the rock sample.

In another embodiment, the hybrid network structure comprises of an input layer, CNN 310 feature maps and DNN 312. Input layer consisting of medical-CT raw image 308 taken as input data, CNN 310 feature maps consisting of convolution, padding and pooling. DNN 312 connects dense layers of multi-layer perceptron (MLP) neural networks, taking CNN 310 features maps as input. The output 314 obtained is a graphical representation of the preferred value vs the actual value. The proposed workflow in FIG. 3 can be applied to other petrophysical properties such as rock elastic properties, relative permeability or capillary pressure provided that enough data is available to train the network.

A large number of reservoir rock samples with their porosity and permeability computations are used for training the hybrid network. The hybrid model predicts the porosity and permeability based only on the medical-CT images 308 of the sample without the need to clean the sample. In accordance with the present embodiment, the POR-PERM (porosity-permeability) predictor engine 304, is built on a deep learning multilayer perceptron (MLP) architecture, which predicts the porosity and permeability of the rock sample based on the PNCE and OOTB 204 feature extraction engines, which then extracts the features from the Micro-CT 302. The POR-PERM predictor engine 304 model is fitted on the training data and validated using various medical CT imaging samples 308 of unclean rock or carbonate core plug samples serving as input to the network, which is scanned at low resolution around 100 um-500 um. At this resolution the pore structure cannot be captured.

Accordingly, the input medical-CT images 308 are first segmented out at a given threshold between Tₒ and q*Tₒ. Threshold, Tₒ, is provided by an automatic segmentation technique such as Otsu's algorithm which overestimates the actual threshold level. The constant q, thresholding factor, is chosen to cover all possible segmentation levels. The segmented binary images generated are used to derive the factious porosity and permeability. The porosity is computed from the binary images and the Pore Network Correction Engine (PNCE) is used to compute the permeability. The Fictious porosity and permeability is computed using the segmented Medical CT images 308. This serves as input to the hybrid network.

Many changes, modifications, variations and other uses and applications of the subject invention will become apparent to those skilled in the art after considering this specification and the accompanying drawings, which disclose the preferred embodiments thereof. All such changes, modifications, variations and other uses and applications, which do not depart from the spirit and scope of the invention, are deemed to be covered by the invention, which is to be limited only by the claims which follow.

1-23. (canceled)

24. A method of detecting a plurality of petrophysical properties of an uncleaned rock sample, the method comprising the steps of:
capturing an image of the uncleaned rock sample;
passing the captured image through a feature extraction engine;
providing an output from the feature extraction engine to a neural network for estimating the petrophysical properties of the uncleaned rock sample; and
displaying the estimated petrophysical properties of the uncleaned rock sample on a display medium.

25. The method of claim 24, wherein the image of the uncleaned rock sample is a three-dimensional (3D) image.

26. The method in accordance with claim 24, wherein the image of the uncleaned rock sample is a computed tomography (CT) image, a micro-CT image or a medical-CT image.

27. The method in accordance with claim 24, wherein the image of the uncleaned rock sample is acquired at either a low resolution or a high resolution.

28. The method in accordance with claim 24, wherein the micro-CT image is captured from a core-flooding equipment through a fluid displacement test.

29. The method in accordance with claim 24, wherein the petrophysical properties comprise porosity, permeability, elastic property, relative permeability or capillary pressure.

30. The method in accordance with claim 24, wherein the feature extraction engine is a porosity-permeability predictor engine.

31. The method in accordance with claim 24, wherein the feature extraction engine is trained using an Out of the Box (OOTB) feature extractor and a pore network correction engine (PNCE).

32. The method in accordance with claim 31, wherein the Out of the Box (OOTB) feature extractor extracts features comprising porosity of the cleaned rock sample, pore volume distributions of the cleaned rock sample and pore size distributions of the cleaned rock sample.

33. The method in accordance with claim 31, wherein the pore network Correction Engine (PNCE) computes permeability of the uncleaned rock sample using a machine-learning algorithm.

34. A process for predicting phase saturation within a reservoir, the process comprising:
capturing an image of a clean porous medium obtained from the reservoir;
passing the captured image through a feature extraction engine; and
providing an output from the feature extraction engine to a neural network for estimating the petrophysical properties of the porous medium;
wherein estimating the petrophysical properties of the porous medium leads to prediction of the saturation of oil within the reservoir.

35. The process in accordance with claim 34, wherein the image of the cleaned porous medium is a three-dimensional (3D) image.

36. The process in accordance with claim 34, wherein the image of the cleaned porous medium is a computed tomography (CT) image, micro-CT image or a medical-CT image.

37. The process in accordance with claim 34, wherein the image of the cleaned porous medium is acquired at either a low resolution or a high resolution.

38. The process in accordance with claim 36, wherein the micro-CT image is captured from a core-flooding equipment through a fluid displacement test.

39. The process in accordance with claim 34, wherein the petrophysical properties comprise porosity, permeability, elastic property, relative permeability or capillary pressure.

40. The process in accordance with claim 34, wherein the feature extraction engine is a porosity-permeability predictor engine.

41. The process in accordance with claim 40, wherein the feature extraction engine is trained using an Out of the Box (OOTB) feature extractor and a pore network correction engine (PNCE).

42. The process in accordance with claim 41, wherein the Out of the Box (OOTB) feature extractor extracts features comprising porosity of the cleaned porous medium, pore volume distributions of the cleaned porous medium and pore size distributions of the cleaned porous medium.

43. The process in accordance with claim 41, wherein the pore network Correction Engine (PNCE) computes permeability of the cleaned porous medium using a machine-learning algorithm.
